(19) United States
(12) Patent Application Publication
(10) Pub. No.: US 2026/0137333 A1
(43) Pub. Date: May 21, 2026

(54) GAZE STABILITY TEST SYSTEMS AND METHODS THEREOF

(71) Applicant: DZ Balance Innovations LLC, Wilmington, DE (US)

(72) Inventors: Adam Thompson-Harvey, Washington, DC (US); Joel Alan Goebel, Saint Louis, MO (US); Sheikh Iqbal Ahamed, River Hills, WI (US); Masud Rabbani, Milwaukee, WI (US); Gia Oscherwitz, Bethesda, MD (US); Shannon Goebel Hoffman, San Jose, CA (US)

(21) Appl. No.: 19/394,658

(22) Filed: Nov. 19, 2025

Related U.S. Application Data
(60) Provisional application No. 63/722,069, filed on Nov. 19, 2024.

(51) Int. Cl. A61B 5/00; A61B 3/00; A61B 3/032; A61B 3/113; A61B 5/11

(52) U.S. Cl. CPC A61B 5/4023; A61B 3/0041; A61B 3/032; A61B 3/113; A61B 5/0002; A61B 5/1114; A61B 5/4803; A61B 5/7465

(57) ABSTRACT

A system and method for assessing gaze stability and vestibular function using a mobile device are disclosed. The system includes a mobile application configured to execute gaze-stability testing protocols, a display module for presenting visual targets, a head-movement and eye-tracking module utilizing real-time video captured via the device's camera, a speech-recognition module for processing verbal responses, and a data-processing module to analyze head-movement and visual-acuity data. The system enables remote patient assessments and includes protocols such as static visual acuity, visual processing, and mobile gaze stabilization tests to evaluate metrics like peak head velocity and visual acuity. Results can be processed in real-time and can be securely transmitted to clinicians for remote evaluation. The disclosed system provides a cost-effective, user-friendly telehealth solution for vestibular function assessment, eliminating the need for specialized equipment or clinical visits.

### CROSS REFERENCE TO RELATED APPLICATIONS

This application claims the benefit of U.S. Provisional Application No. 63/722,069, filed Nov. 19, 2024, hereby incorporated by reference in its entirety.

### FIELD OF DISCLOSURE

The instant disclosure relates to telehealth systems and methods for remote medical testing, for example, mobile gaze stability testing and other vestibular tests.

### BACKGROUND

Existing tablet-based systems can monitor home gaze stability exercises. While their research demonstrated the feasibility of head and eye gaze detection using the front camera of a tablet-based mobile device, their clinical outcome measures consisted of consistency in performing a VORx1 exercise at an assigned head velocity (a common vestibular rehabilitation exercise) while maintaining gaze on a stationary target (an “X”). Additionally, the existing systems require the subject to position the device at least 1 meter from the subject and does not address the target size issues related to dynamic visual acuity.

Thus, there is a need in the industry to improve the simplicity by which gaze stabilization data is obtained by utilizing a single target size and measuring peak head velocity achieved for target recognition as the outcome measure of dynamic visual acuity.

### SUMMARY OF THE INVENTION

In an embodiment, a system for assessing gaze stability and vestibular function using a mobile device is provided. The system includes a mobile application configured to execute gaze-stability testing protocols, a display module for presenting visual targets on a screen of the mobile device, a head-movement and eye-tracking module utilizing real-time video captured via a camera of the mobile device, a speech-recognition module configured to process verbal responses, and a data-processing module that analyzes head-movement and visual-acuity data to determine gaze-stability metrics.

In some embodiments, the head-movement and eye-tracking module can use augmented-reality frameworks to calculate positions and distances of at least one of a user's head or eye. The gaze-stability testing protocols can include a static-visual-acuity test to establish baseline visual acuity, a visual-processing test to determine minimum display-time thresholds, and a mobile gaze-stabilization test to evaluate a maximum head velocity at which visual acuity is maintained. The display module can dynamically adjust at least one of a size and an orientation of each visual target based on user performance during testing.

In some embodiments, the system can further include a telehealth interface that enables clinicians to access real-time test results. The system can further include a telehealth interface that enables clinicians to guide treatment remotely. The mobile application can implement a modified threshold determination procedure to determine thresholds for both visual acuity and head velocity. The speech-recognition module can use native mobile device frameworks to process verbal responses regarding target orientation. In some embodiments, the display module is configured to present a visual target at a randomized onset time within a velocity-gated window and for a presentation duration equal to a visual display time derived from a visual processing test. The display module can dynamically scale a visual target based on a measured device-to-eye distance and device display characteristics. The device-to-eye distance can be estimated by averaging distances from the camera to each eye derived from face-tracking geometry. The mobile application can be configured to perform at least one of functional vestibular function testing measurements, dynamic visual acuity testing, oculomotor testing measurements, and collection of patient-reported outcomes related to vestibular function.

In an embodiment, a method for evaluating vestibular function using a mobile device is provided. The method includes displaying a visual target on a screen of the mobile device, guiding a user through head-movement exercises while capturing a real-time video stream of the user, tracking head and eye movements using the video stream, collecting verbal responses via a speech-recognition module, analyzing head-movement and visual-acuity data to determine gaze-stability metrics, and transmitting test results for remote evaluation.

In some embodiments, the visual target can be a dynamically adjustable optotype that is displayed for a duration based on user performance in prior tests and whose size is preset to a value relative to the user's baseline static visual acuity. The head-movement and eye-tracking module can use augmented-reality frameworks to calculate positions and distances of at least one of a user's head or eye. The head-movement exercises can include at least one gaze-stability testing protocol including a static-visual-acuity test to establish baseline visual acuity, a visual-processing test to determine minimum display-time thresholds, and a mobile gaze-stabilization test to evaluate a maximum head velocity at which visual acuity is maintained. The method can further include dynamically adjusting at least one of a size and an orientation of each visual target based on user performance during testing. The method can further include implementing a modified threshold determination procedure to determine thresholds for both visual acuity and head velocity of the user.

In some embodiments, the method can further include presenting the visual target at a randomized onset time within a velocity-gated window and for a presentation duration equal to a visual display time derived from a visual processing test. The method can further include dynamically scaling a visual target based on a measured device-to-eye distance and device display characteristics. The method can further include at least one of functional vestibular function testing measurements, dynamic visual acuity testing, oculomotor testing measurements, and collection of patient-reported outcomes related to vestibular function.

There are no current FDA-approved mobile applications for home vestibulo-ocular reflex monitoring, thus the instant disclosure provides for technology that can allow for a user-friendly, smartphone-based ability to track head velocity with minimal supervision to obtain vestibular function information remotely. The disclosed advantageously improves the simplicity by which gaze stabilization data is obtained for telehealth purposes. In some embodiments, nothing more than the patient's smartphone and direct human-device interaction are required whereby all relevant measures are integrated and recorded using the same application at a natural position (i.e., smartphone use) that can be performed anywhere. In some embodiments, the instant systems and methods can assess head velocity and gaze fixation due to the contribution of the patient's vestibulo-ocular reflex that equals measures of gaze stability obtained by validated, in-office computerized assessments (the Gaze Stabilization Test).

This disclosure will now provide a more detailed and specific description of the accompanying drawings. The drawings and specific descriptions of the drawings, as well as any specific or alternative embodiment discussed, are intended to be read in conjunction with the entirety of this disclosure. These embodiments are provided by illustration only so that this disclosure will be thorough, complete, and fully convey understanding to those familiar with the field.

### BRIEF DESCRIPTION OF THE DRAWINGS

FIG. 1 illustrates a schematic view of a device for use with the present systems and methods according to an embodiment;

FIG. 2 illustrates a schematic view of a gaze stabilization test according to an embodiment;

FIGS. 3A-3L illustrate various example implementations of software according to an embodiment; and

FIG. 4 illustrates a method of use according to an embodiment.

### DETAILED DESCRIPTION

In the USA, more than a third of adults over 40 years of age suffer from dizziness secondary to vestibular dysfunction. Existing practices recommend vestibular rehabilitation therapy where additional sensors or devices are used. For patients undergoing home therapy, current real-time tele-assessments of vestibular disorder patients remain a challenge due to the need for extra equipment setup. Although notable attempts have been made to utilize virtual reality and mobile-based technology to monitor postural balance and head motion, recent advances in native smartphone-based technology provide us with the capability to develop a simple, patient-friendly approach to assessing vestibular dysfunction via the vestibulo-ocular reflex (VOR). The instant system and methods can fill a need in a crucial area of telehealth in vestibular-related health care by using existing mobile phone technology and the well-established Gaze Stabilization Test protocol to cost-effectively assess vestibular dysfunction in patients remotely. In some embodiments, the instant system and methods can allow clinicians to receive real-time assessments to guide treatment.

The instant disclosure addresses the significant challenge of providing accessible, cost-effective, and accurate vestibular function assessments for patients experiencing vestibular dysfunction, which often results in dizziness, imbalance, and impaired vision during head movements. Traditional methods for conducting Gaze Stabilization Tests (GST) rely on expensive, clinic-based equipment and trained personnel, restricting their availability to specialized facilities. This disclosed approach overcomes these limitations by utilizing smartphone technology to offer a mobile, user-friendly platform capable of performing GST and related vestibular assessments remotely, without requiring additional hardware. The instant system can employ a smartphone-based application that can integrate native augmented reality frameworks (e.g., iOS ARKit, Android ARCore), computer vision algorithms, and speech recognition modules to guide users through a series of vestibular tests. These tests can include the Static Visual Acuity (SVA) test, Visual Processing Test (VPT), and Mobile Gaze Stabilization Test (mGST), which collectively measure the user's ability to maintain visual focus during controlled head movements. In some embodiments, the system can dynamically adjust target size based upon the distance between the user and the device as well as adjust the display time and head velocity thresholds based on user performance, ensuring accurate and personalized assessments. In some embodiments, results, such as peak head velocity and visual acuity metrics, can be processed in real-time and can be securely transmitted to clinicians for remote evaluation. In some embodiments, the instant system and methods can be configured to allow services such as telehealth integration for clinician-guided sessions and optional AI-assisted supervision for enhanced diagnostic accuracy. This described approach significantly expands access to vestibular care, enabling patients to perform reliable assessments at home while maintaining clinical-grade accuracy.

For purposes of this disclosure, the term “module” refers to a software, hardware, or firmware component, or any combination thereof, configured to perform one or more specified functions. A module may be implemented as a set of executable instructions stored in a non-transitory computer-readable medium and executed by one or more processors, as dedicated hardware circuitry, or as a combination of hardware and software. The term “application” refers to a set of computer-executable instructions, routines, or code segments that, when executed by a processor of a mobile device, perform the specified operations described herein.

As used herein, the terms “configured to,” “programmed to,” or “operable to” mean that the referenced component, module, or application is implemented, programmed, or otherwise arranged to perform the recited function, either directly or through interaction with other components or modules. The term “computer-readable medium” includes all forms of memory, storage, and transmission media capable of storing or conveying instructions for execution by a processor, including but not limited to RAM, ROM, flash memory, hard drives, optical media, and network transmission signals.

The systems and methods described herein may be implemented either entirely or partially as software executed on one or more processors of a mobile device 100, such as a smartphone, tablet, or other programmable device. The software may be stored in a non-transitory computer-readable medium and may be distributed via application storefronts, direct download links, or other electronic means. The described modules and applications may be implemented using any suitable programming language or framework, including but not limited to native mobile operating system frameworks, augmented reality libraries, computer vision toolkits, and speech recognition engines. All references to “executing,” “processing,” “analyzing,” “presenting,” or similar terms in the claims and specification are intended to encompass operations performed by software, hardware, or firmware, or any combination thereof, unless otherwise specified.

Disclosed herein is a computerized process consisting of a smartphone-based mobile application platform that uses native computer vision smartphone capabilities and the Gaze Stabilization Test protocol as a diagnostic and rehabilitation tool to cost-effectively track and assess vestibular dysfunction patients remotely while allowing patients to perform gaze exercises with a stable visual background. Furthermore, clinicians receive a real-time assessment to guide further treatment.

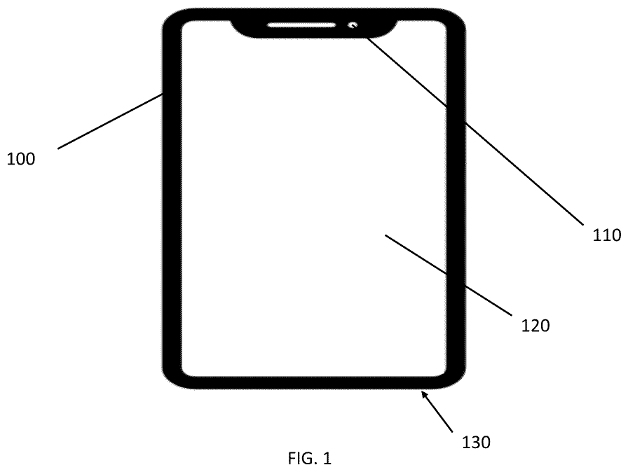

In some embodiments, as shown in FIG. 1, the system may include a device 100 including an on-device module set including: (i) a head-pose and eye-tracking module 110 (e.g., a camera or image sensor, a front facing camera on a smart phone/tablet) configured to derive head kinematics and ocular metrics using one or more of augmented-reality frameworks, computer-vision models, and inertial sensors; (ii) a stimulus control module (e.g., a display) 120 configured to present visual targets and control target timing, duration, and size; (iii) a speech-recognition module 130 (e.g., a microphone and associated software) configured to acquire verbal responses and map the responses to a constrained lexicon of orientation terms; (iv) a quality-assurance and safety module configured to monitor test conditions, including device-to-eye distance, head excursion limits, tracking confidence, and participant safety criteria; and (v) a data-handling module configured to compute and store derived metrics locally and, with user consent, to transmit results to a secure network service. In some embodiments, a network service may provide authentication, encrypted data synchronization, and access to a clinician-facing portal for visualization, report generation, and alerts.

In some embodiments, the system may combine inertial measurement unit (IMU) data from the device 100 (for example, gyroscope and accelerometer data sampled at or above approximately 100 Hz) with pose estimates derived from an augmented-reality framework to estimate head angular velocity (e.g., for use in a gaze stabilization test) and peak head velocity with improved temporal fidelity. The system may implement a calibration procedure to estimate and compensate for static bias and scale factors of the gyroscope, optionally using short-duration still periods or reference poses detected by computer vision. In some embodiments, a complementary filter or Kalman filter may fuse high-rate gyroscope integration with lower-rate, drift-resistant head orientation derived from computer vision to provide stable estimates of instantaneous head velocity and peak. In some embodiments, when face-tracking confidence falls below a threshold, the system may temporarily fall back to IMU-only estimates and flag the trial quality for later review.

In an embodiment, the instant system can employ a number of modules, or processes, for example computer processes, that can be made up of the following executable modules: 1) iOS ARKit framework; 2) Android ARCore; 3) iOS SceneKit; 4) iOS Speech framework; 5) Android SpeechRecognizer; 6) Gaze Stabilization Test protocol; 7) a device 100 (e.g., a smartphone, a tablet, a smart TV, AR/VR goggles, heads up displays, or other programable devices as are known in the art); 8) a display 120 on the target device 100; 9) measurement of detecting the head and eyeball movements; and 10) eyeball tracking from real-time facial video.

In some embodiments, the system may employ native augmented reality frameworks on the device 100 to provide a primary platform for real-time facial tracking. For iOS devices, ARKit may be utilized, and for Android devices, ARCore may be utilized. The real-time facial tracking may be configured to determine head movement parameters, including angular displacement and velocity, and may extract eye-tracking metrics indicative of gaze direction and fixation, thereby enabling assessment of head motion and ocular behavior during testing.

In some embodiments, an augmented reality framework on the device 100 may utilize available face-tracking capabilities to enable real-time detection and tracking of facial features. For iOS devices equipped with a TrueDepth camera system, ARKit may be employed to determine whether face tracking is supported and to establish a face-tracking configuration. Upon detection of a face, the framework may generate an anchor object representing facial geometry, which may be used to derive head pose and ocular feature locations. For Android devices, ARCore may be utilized to provide analogous functionality. Eyeball tracking may be performed using geometric transformations that may include translation, expressed as x′=x+dx, y′=y+dy, z′=z+dz; scaling, expressed as x′=sₓ×x, y′=s_{y}×y, z′=s_{z}×z; and rotation about an axis, such as a Z-axis rotation expressed as x′=x×cos(θ)−y×sin(θ) and y′=x×sin(θ)+y×cos(θ).

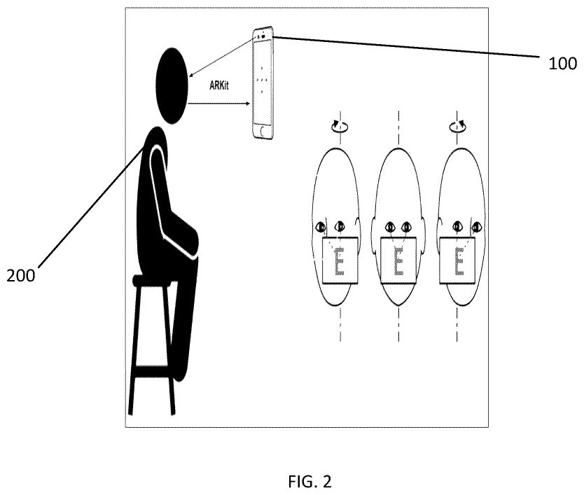

In some embodiments, for example when using iOS devices, a scene rendering framework (e.g., iOS SceneKit) may be employed in conjunction with the augmented reality face-tracking framework to determine and maintain a predefined separation distance between the smartphone and the user's 200 face, as shown in FIG. 2, such as a minimum distance of approximately 50 centimeters. Maintaining this device-to-face distance may be configured to support accurate execution of the Gaze Stabilization Test and adherence to applicable clinical or medical standards. As will be discussed further below, the instant disclosure provides for users locating themselves any distance beyond the 50 cm minimum. In some embodiments, a scene-rendering framework (for example, SceneKit on iOS) may be employed in conjunction with the augmented reality framework to determine a distance between the user's face and the device 100. The system may compute distances from the device's camera 110 to each eye and may utilize an average of those distances to estimate a head-to-device 100 separation. For Android implementations, ARCore may be used in place of ARKit to provide comparable distance estimation functionality.

In some embodiments, a speech-recognition framework on the device 100 can enable capture and processing of participants' verbal responses. For iOS devices, the iOS Speech framework may be utilized. For example, with iOS implementations, the iOS Speech framework may be employed, and an SFSpeechRecognizer may be configured to convert spoken input to text, which may then be compared against a predefined set of orientation terms. For Android devices, the Android SpeechRecognizer may be utilized. For example, a SpeechRecognizer may be used to provide analogous speech-to-text functionality, and associated processing operations may be implemented similarly to those of the iOS application. These frameworks may operate in coordination with the display 120 and a microphone 130 on the target device 100 to capture vocal responses during a visual acuity test that forms part of the Gaze Stabilization Test protocol. Such verbal responses can include noting the direction of a target, e.g., the letter “E.”

In some embodiments, a Gaze Stabilization Test protocol may be implemented to obtain peak head velocity, expressed in degrees per second, and visual acuity with the head both stationary and moving, measured in log MAR and Δ log MAR, respectively, using the instant application. The protocol may be configured to identify the highest peak head velocity at which the user may maintain visual acuity, using only a camera on device 100 such as the front facing camera 110 so the screen 120 remains visible to the user, as shown in FIGS. 1 and 2. The protocol may coordinate measurement of head movement and eye tracking from real-time facial video in a sequence that may include: (i) a Static Visual Acuity assessment, (ii) a Visual Processing Test (VPT), and (iii) a Mobile Gaze Stabilization Test (mGST). The instant application can be installed on device 100 using existing application storefronts, or via specialized links provided to the user. The device 100 can be used to provide input to the instant application as input for the above noted protocols. The device 100 can additionally include a display for displaying a target, e.g., the letter “E,” on the display for the various tests.

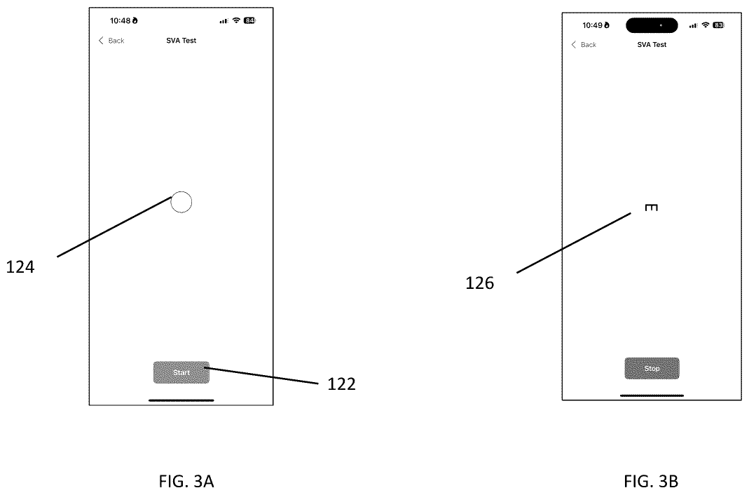

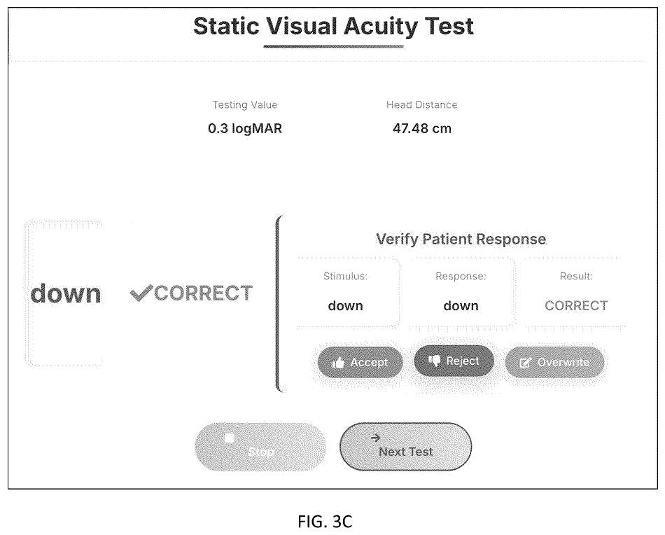

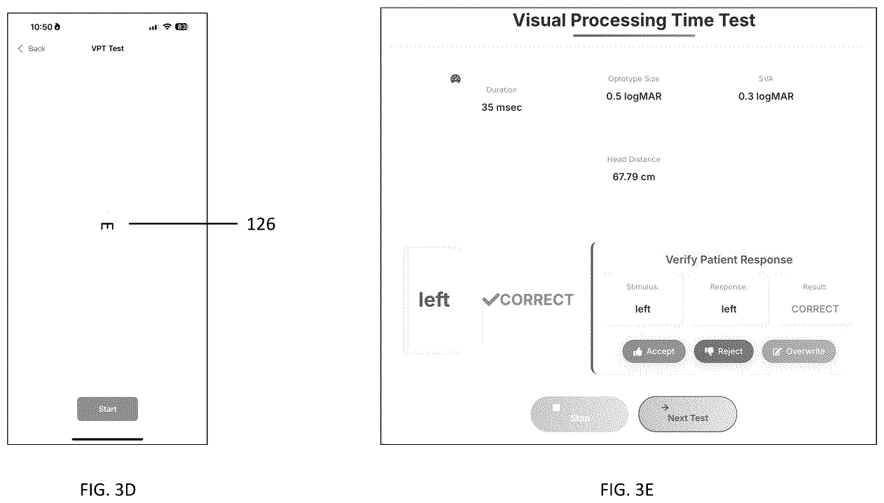

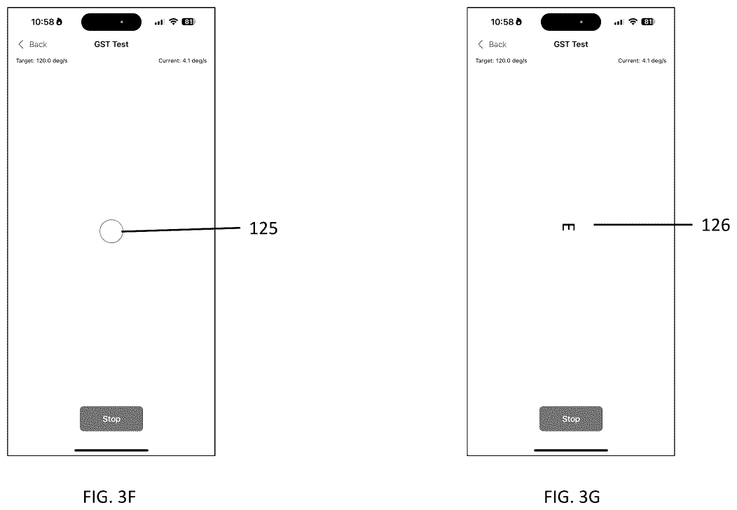

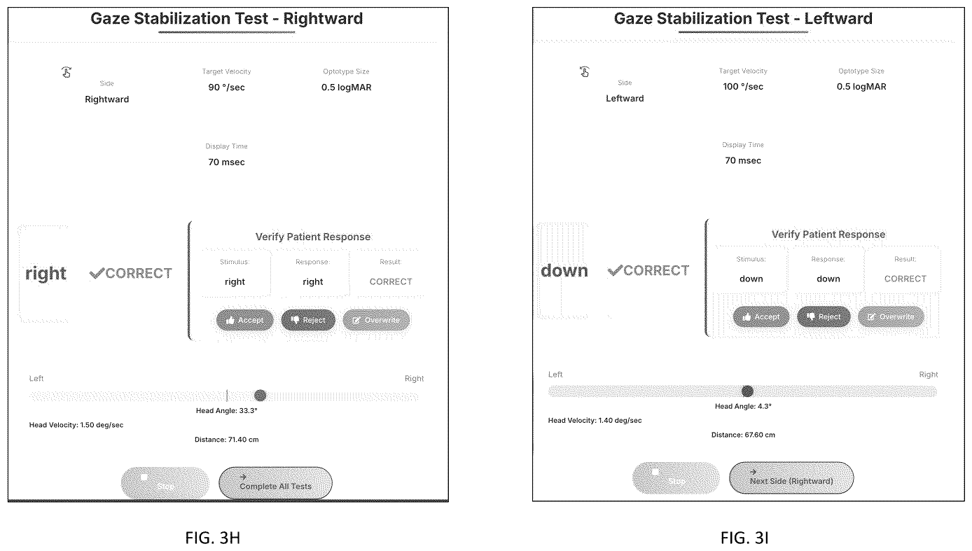

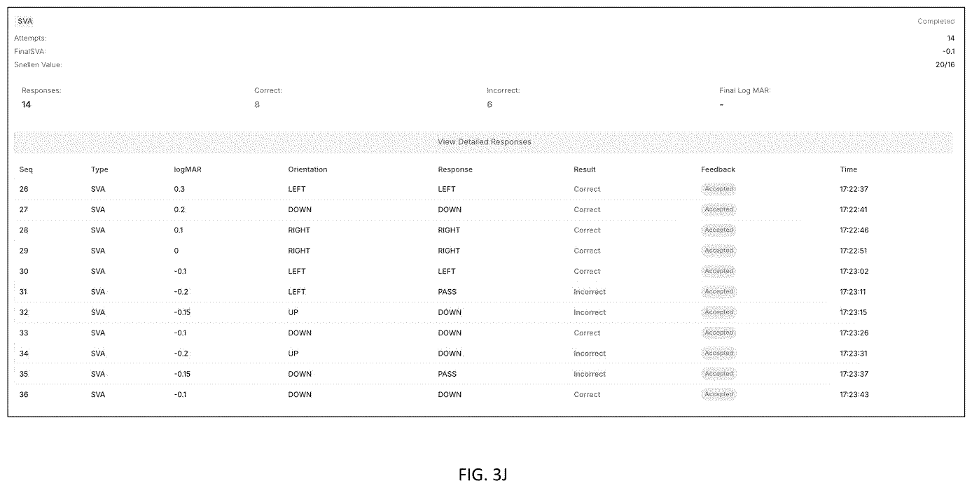

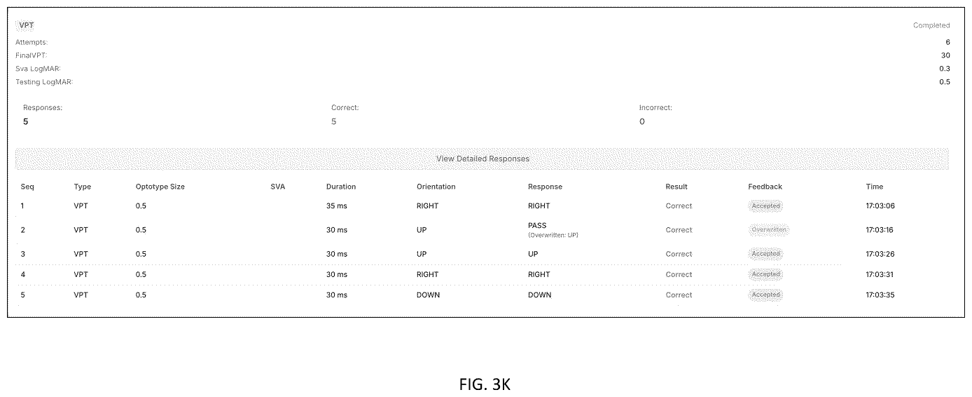

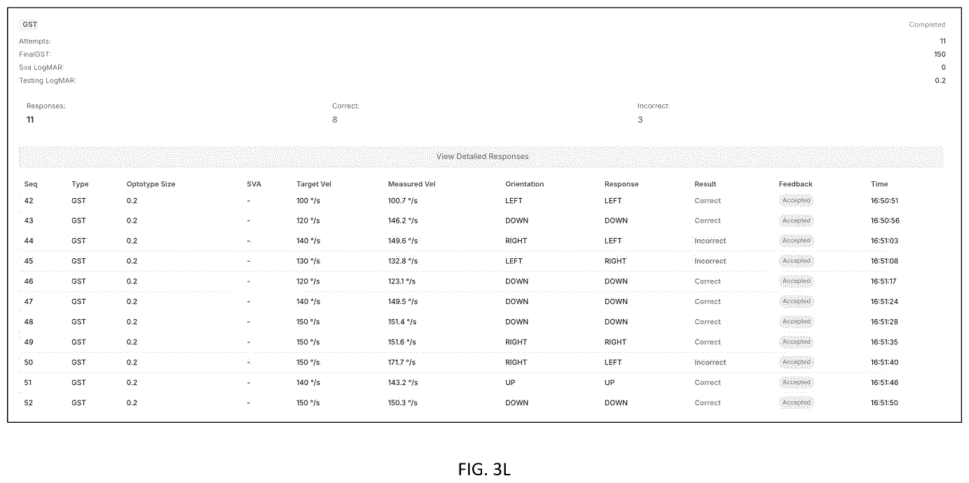

In some embodiments, a Static Visual Acuity (SVA) procedure may be implemented to establish a baseline visual acuity for a participant, which may determine a target size for use in subsequent tests. The participant may be instructed to maintain gaze on the device 100 screen while positioning the mobile device 100 on a stand approximately 0.5 meters from the participant with the head stationary. When head placement and distance are within acceptable ranges, the device's display 120 can indicate readiness (e.g., showing a green “Display” button 122), and the participant may initiate the procedure by activating a start control, e.g., button 122, as shown in FIG. 3A. Prior to a first trial, on-screen instructions may be presented to describe target orientations, such as up, down, left, and right, for an optotype 126 (for example, the letter “E”) that may appear on the screen, as shown in FIG. 3B. The instructions may advise the participant to verbalize the perceived target orientation.

During each trial, the participant may be prompted to fixate on a central fixation element 124 for a predetermined period (for example, approximately 2 seconds). The fixation element 124 may then be removed, and a target optotype 126 may be presented at the fixation location for a brief duration (for example, approximately 1.5 seconds), after which the participant may report the perceived orientation aloud. Native speech-recognition services of the device 100 may be enabled to record and process the participant's response. Based on the participant's response, the target size may be adaptively adjusted, as will be described below, until a static visual acuity threshold is determined using a computer-implemented modified threshold determination procedure. In some embodiments, the modified threshold determination procedure may comprise increasing the target size by 0.05 log MAR for incorrect or non-responses and decreasing the target size by 0.10 log MAR for correct responses until the participant correctly identifies the target orientation in three out of five presentations. The application may repeat a given trial when speech recognition is unable to decipher the response or detects words outside a predefined set of orientation terms. For subsequent testing within a gaze stabilization protocol, the target size may be set to a value relative to the participant's static visual acuity, such as approximately 0.2 log MAR above the determined SVA (e.g., about two lines larger than a 20/20 equivalent on a Snellen chart). In some embodiments, a clinician can be remotely monitoring (e.g., ensuring the stimulus matches the response from the user 200) and controlling the test being performed on the user's device 100, as shown in FIG. 3C. In some embodiments, the SVA value can determine the size of the target of the subsequent steps.

In some embodiments, a Visual Processing Test (VPT) may be implemented to determine a shortest display time at which a participant can accurately recognize a target 126 orientation, as shown in FIG. 3D. The participant may again be prompted to fixate on a central fixation element on the application screen for approximately 2 seconds. In some embodiments the central fixation element, or target 126, can be sized on the screen based on the SVA value. The fixation element 126 may then be removed after an interval, such as approximately 250 milliseconds. A target 126 (for example, a letter “E”) sized at approximately 0.2 log MAR above the participant's static visual acuity may then be presented at the fixation location for a defined duration (for example, approximately 250 ms), and the participant may report the perceived orientation aloud. Native speech-recognition services of the mobile device 100 may be enabled to record and process the participant's response.

Additional VPT trials may be conducted with targets presented at progressively shorter presentation times until a minimum threshold is determined using a modified threshold determination procedure. In some embodiments, the modified threshold determination procedure may comprise increasing the presentation time by approximately 5 ms for incorrect or non-responses and decreasing the presentation time by approximately 10 ms for correct responses until the participant correctly identifies the target orientation in three out of five presentations. In some implementations, the minimum threshold may not exceed approximately 70 ms. In some embodiments, the VPT test can start at 35 ms and then increase the time (for incorrect answers) or decrease the time (for correct answers) by 5 ms. There is little clinical value to going below 30 ms; so if the user gets the orientation of the target correct at 30 ms, the user may test again at 30 ms. In some embodiments, when the user gets three correct answers over three incorrect answers the VPT is calculated. However, as there is little to no clinical value, there may be no need to test below 30 ms. As such, when the user gets three correct answers at 30 ms, this can be the only case where the user would not need three wrong answers at a lower time to determine value.

Once a VPT minimum threshold is determined, a Visual Display Time (VDT) may be computed as VDT=VPT+35 milliseconds, and the VDT may be used as a target display-time parameter for subsequent testing. In some embodiments, the subroutine output may include the VPT value representing the minimum display time at which the participant can consistently recognize the target, and the corresponding VDT derived by adding the buffer. In some embodiments, a clinician can be remotely monitoring (e.g., ensuring the stimulus matches the response from the user 200) and controlling the test being performed on the user's device 100, as shown in FIG. 3E.

In some embodiments, a Mobile Gaze Stabilization Test (mGST) may be implemented to determine a maximal head velocity during active head rotations in the horizontal (yaw) plane at which a participant may maintain visual acuity at a level set relative to static acuity (for example, approximately 0.2 log MAR above baseline). The participant may be prompted to fixate on a central fixation element 125 on the application screen for approximately 2 seconds and pause and stop controls may be available throughout, as shown in FIG. 3F. Upon initiation, the participant may move the head side to side while progressively increasing movement speed, for example at a pace set by a metronome sound provided by the device 100 via the application. A visual target 126 (for example, a rotated letter “E”) may be presented when an expected head-velocity threshold for a given trial is reached, as shown in FIG. 3G.

At the start of mGST testing, an initial lower and upper head-velocity bounds may be set (for example, approximately 60 degrees per second and approximately 150 degrees per second, respectively). The software may randomly select an initial target head velocity for the participant to attempt to achieve. Once the target head velocity is achieved, the target may be displayed at a size determined by the Static Visual Acuity (SVA) procedure for a duration determined by the Visual Display Time (VDT) calculated in the Visual Processing Test (VPT).

For subsequent trials, the target velocity may be determined based on prior performance. In some embodiments, if the participant correctly identifies the target orientation at the lower bound speed, the lower bound may be increased by approximately 20 degrees per second for a subsequent trial, though other increases of time are possible. If the participant fails to identify the orientation at the lower bound speed, the upper bound may be set to the current lower bound value, and the lower bound may be reduced by approximately 20 degrees per second, after which the participant may proceed with testing at the upper bound speed. If the participant correctly identifies the orientation at the upper bound speed, the lower bound may be set to the current upper bound value, and the upper bound may be increased by approximately 20 degrees per second for a subsequent trial. If the participant fails at the upper bound speed, the upper bound may be reduced by approximately 20 degrees per second. This iterative upper-lower bound adjustment may continue until the difference between bounds is minimized, thereby determining a maximum achievable head velocity threshold while maintaining visual acuity. The mGST can be controlled and monitored from the website and real time participants' response and state of the test can be controlled and manipulated from website, as shown in FIGS. 3H and 3I.

Target presentation time may be set to equal the VDT determined in the VPT, and head excursions may be constrained such that net azimuth does not exceed approximately 15 degrees from midline in each direction. The test may be deemed complete when the participant correctly identifies target orientations in three out of five presentations at a peak target velocity determined by the protocol, such as by a modified threshold determination method. In some embodiments, the subroutine output may include an mGST score representing the highest head velocity at which the participant can correctly identify target orientation.

In some embodiments, to reduce anticipatory responses and to elicit reflex-driven performance, the stimulus control module may randomize target onset within a velocity-gated window. For example, once head angular velocity enters a pre-defined band centered on a target velocity and head excursion remains within +15 degrees of midline, the target may be presented after a randomized delay selected from a bounded distribution (for example, approximately 50-250 milliseconds), while ensuring that the resulting presentation duration equals the VDT determined by the VPT. In some embodiments, inter-stimulus intervals and target orientations may be pseudo-randomized, and spatial jitter within a constrained fixation region may be applied to reduce predictability.

In some embodiments, the system may compute auxiliary metrics including: (i) perception time test (PTT), defined as a time interval from target onset to a valid verbal response recognized by the speech module; (ii) directional asymmetry, defined as a difference or ratio between leftward and rightward mGST thresholds; (iii) confidence scores for each trial based on tracking confidence, head-excursion compliance, and speech-recognition certainty; and (iv) comparison of outcomes to age-stratified normative datasets, producing categorical labels or flags (for example, “within expected range,” “below expected,” or “significantly below expected”). In some embodiments, the system can provide for symptom scoring, e.g., a dizziness symptom questionnaire, to provide a more holistic view of the patient's condition.

In some embodiments, prior to testing, a safety checklist may be presented recommending seated posture, adequate lighting, and a clear environment. During testing, the system may monitor real-time indicators, including excessive head excursion beyond ±15 degrees, loss of tracking, or reports of dizziness, and may automatically pause or present a prompt advising the participant to rest. In some embodiments, pause and stop controls may remain available throughout the protocol, and the system may record reasons for aborted sessions. With consent, the system may notify a designated clinician when a session is repeatedly aborted for safety-related reasons.

In some embodiments, a tutorial mode may guide the participant through distance calibration, fixation practice, and sample trials at low speed, with real-time feedback on head excursion and distance. The system may present progress indicators (e.g., color codes to provide users with immediate feedback), trend summaries, and motivational messages (for example, improvements in mobile GST compared to baseline). For example, the system can create a “gamification” of the testing by providing a motivational message along the lines of, “beat your high score.” In some embodiments, configurable reminders or nudges may encourage adherence to prescribed testing schedules via notifications provided to the user's personal devices.

In some embodiments, a clinician may remotely initiate a supervised session, adjust testing parameters in real time (for example, target size offset relative to SVA, VDT, initial velocity bounds, step sizes, and randomization ranges), and observe quality indicators including tracking confidence, device-to-eye distance, and head excursion compliance. In some embodiments, a clinician can observe summarized results of the SVA, VPT, and mGST, as shown in FIGS. 3J, 3K, and 3L. In some embodiments, tele-video may be optionally enabled for live observation while preserving on-device processing of raw sensor data. For example, a clinician can initiate an mGST during a remote visit, review any asymmetry/PTT, and can then set follow-up cadence for the patient. In some embodiments, a patient-provider linking workflow may employ secure invite codes or pairing requests, after which results may appear in a clinician's roster subject to ongoing consent. The portal may provide patient list management, session histories, and configurable sharing scopes.

In some embodiments, a clinician-facing portal may present longitudinal charts of SVA, VPT, VDT, mobile GST thresholds, PTT, and asymmetry metrics, with overlays of age-normative ranges. In some embodiments, authorized users may view and export detailed derived data per session (e.g., per-trial head velocity, correctness, response latency, tracking confidence) and, where available, eye-movement feature traces, while raw video/audio are not retained, with or without raw video frames or audio signals. The portal may provide automated report generation summarizing results, interpretations based on configurable clinical rules, and clinician-editable notes. In some embodiments, the reports or structured results can be uploaded to an electronic health record system to generate usage logs suitable for billing documentation for billing workflows. In some embodiments, alerts may be generated when performance declines beyond a threshold, when adherence falls below a prescribed schedule, or when quality indicators suggest invalid results. Role-based access control and audit logging may be provided to ensure compliance with patient data privacy requirements. In some embodiments, the system can be configured to align with applicable privacy and security standards (e.g., HIPAA) including access controls, audit logs, minimum necessary data handling, and breach notification procedures.

In some embodiments, raw video frames and raw audio signals may be processed on-device 100 in real time and may not be retained after feature extraction, with only derived metrics (for example, peak head velocity, response correctness, timing, and quality indicators) being stored, for example to be maintained in a medical record. Alternatively, rad video frames, raw audio signals, and other collected data may be made available to the clinician. In some embodiments, the device 100 may cache the data locally in an encrypted queue when a network is not available and automatically synchronize data once prompted or when a network connection is reestablished. In some embodiments, data stored on the device 100 may be encrypted, and any data transmitted to network services may be encrypted in transit and at rest. The application may implement explicit consent flows to enable or disable data sharing with identified clinicians, and users may revoke consent at any time to stop future synchronization.

In some embodiments, machine-learning models may monitor real-time signals to score trial quality, detect compensatory eye movements, or suggest repeating a trial when confidence is low. In some embodiments, predictive analytics may estimate fall risk or likelihood of vestibular hypofunction from longitudinal trends in mobile GST, PTT, and asymmetry, optionally adjusting recommended testing cadence. In some embodiments, the instant system can collect data to be fed into a machine-learning model to enhance the veracity of the model as more data is collected. In some embodiments, system performance may be validated against a reference inertial measurement unit affixed to the head, and agreement within a predefined margin (for example, mean absolute error or Bland-Altman limits) may be recorded. In some embodiments, software may flag outlier sessions for clinician review. In some embodiments, a research mode may enable export of de-identified derived metrics with configurable fields (for example, age bands, device 100 class, test outcomes) and may enforce governance controls including access approvals and audit trails to support study compliance.

In various embodiments, the mobile application platform may additionally be programmed to produce optional executable steps, which may include: functional vestibular function testing measurements; dynamic visual acuity testing; oculomotor testing measurements; collection of patient-reported outcomes related to vestibular function; AI-assisted testing supervision and predictive capabilities; and real-time, tele-video clinician supervision. In some embodiments, the system may implement additional test modules, including: (i) a horizontal-plane video head impulse test (vHIT) configured to estimate vestibulo-ocular reflex gain by analyzing eye response to brief head impulses; (ii) spontaneous nystagmus detection with and without visual fixation; (iii) saccade testing to measure latency, peak velocity, accuracy, and conjugacy using randomized target jumps; (iv) gaze-evoked nystagmus assessment at eccentric gaze positions; (v) fixation-suppression testing during visual pursuit; (vi) skew deviation detection using a vertical cover test; (vii) positional nystagmus evaluation during maneuvers including Dix-Hallpike and supine roll; and (viii) postural sway analysis using inertial sensors to estimate sway metrics under various stance conditions.

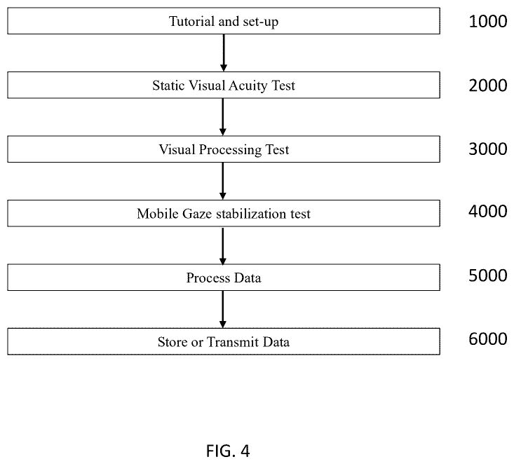

In an embodiment a method of use is provided, as shown in FIG. 4. In some embodiments, an initial setup procedure, Step 1000, may be implemented to prepare a smartphone for testing. Advantageously, the instant systems and methods replicate the core functions of a clinical gaze stabilization test using a single camera, e.g., a smartphone camera, without the need for clinical instrument measurements. The smartphone may be placed on a stable mount at approximately 0.5 meters from the participant's face, and the application may determine and maintain this distance throughout the test using input from the camera. The device 100 may be aligned with the participant's 200 face at eye level to support optimal tracking, as shown in FIG. 2. The application may be installed and opened to begin a Gaze Stabilization Test (GST) protocol, and the participant may provide consent for access to the camera and microphone to enable head and eye tracking and speech recognition. The participant may be advised that the test may comprise three parts and that on-screen instructions may be followed throughout.

As noted above, the device 100 may not be at a predetermined, or fixed, distance from the participant. For example, the user may hold the device 100 at arm's length or otherwise places the device 100 at eyelevel. As such, the instant systems and methods provide a module that can adjust the relative size of a target to ensure consistency in measured values during the tests. In some embodiments, the system may determine a device-to-eye distance using one or more of: stereo depth from a TrueDepth camera, monocular depth from facial geometry anchors, or device-reported focus distance proxies, and may compute an average of distances from the camera to each eye to estimate a head-to-device separation. The display module may compute an optotype's physical size required to subtend a desired visual angle at the measured distance and may convert the physical size to device-specific units using pixel density and scale factors. In some embodiments, the application may support distances greater than approximately 50 centimeters by proportionally scaling optotype size to maintain the intended visual angle, while enforcing a minimum separation distance to ensure accurate measurements.

In some embodiments, optotype sizing may be determined using standard visual acuity conventions. As the instant system and method is device 100 agnostic, the software can adjust the size of the optotype to ensure consistency with the testing using screens having different sizes and pixel density, or different distances between the device 100 and the user. At 0.0 log MAR (Snellen 20/20), a standard optotype (for example, a letter “E”) may be defined such that the entire letter subtends 5 arcminutes at the eye, with each stroke subtending approximately 1 arcminute; equivalently, the minimum angle of resolution (MAR) may be 1 arcminute and log MAR may equal 0. In practice, at a viewing distance of approximately 6 meters, a 20/20 optotype may be about 8.7 millimeters tall. In some embodiments, the optotype may be rendered with a square bounding box (for example, a 5:5 height-to-width ratio), such that setting the physical height may inherently set the correct width.

In some embodiments, to display a 0.0 log MAR optotype at approximately 50 centimeters, the optotype may be sized to subtend 5 arcminutes at that distance. For example, a total visual angle θ can be approximately 5 arcminutes; the physical height H at distance D may be given by geometry as:

$$H = 2 D tan ( \frac{θ}{2} ) .$$

In some embodiments, the device 100 can calculate the distance D between the user and the device 100. For calculations in display units, distance may be converted to inches to align with pixels-per-inch (PPI). For example, at D=50 cm, Dᵢₙ≈50/2.54 inches. With a half-angle of approximately 2.5 arcminutes, tan (θ/2) may be approximated for small angles, yielding a physical height Hᵢₙ on the order of 0.0286 inches (approximately 0.727 millimeters). Accordingly, the optotype may be rendered at this physical height to subtend the intended angle at 50 centimeters, and, due to the square bounding box, the physical width may match the height. This process can be completed before each test performed by the device 100.

In some embodiments, for example with iOS devices, physical dimensions may be converted to resolution-independent points using device 100 display characteristics. Pixels per inch (PPI) may denote pixel density (for example, approximately 460 ppi), and a scale factor S (for example, 2× or 3×) may relate pixels to points via pixels=points×S. Given a physical height Hᵢₙ, a pixel height Nₚₓ may be computed as Nₚₓ=Hᵢₙ×PPI, and an iOS point size may be computed as Points=Nₚₓ/S. Combining these, a required point size P may be expressed as:

$$P = \frac{2 ( \frac{D}{2.54} ) tan ( {2.5}^{'} ) \times P P I}{S} ,$$

where D is in centimeters, PPI is pixels per inch, S is the device 100 scale factor, and 2.5′ denotes 2.5 arcminutes converted to radians for the tangent operation. Numerically, for D=50 cm, PPI≈460, and S≈3, the point size may be approximately 4.39 points. Thus, an optotype of about 4.4 points in height (and width) may subtend approximately 5 arcminutes at 50 centimeters on an iPhone-class display with those parameters.

In some embodiments, the foregoing relationship may be summarized as:

$$PointSize \approx \frac{2 ( \frac{D}{2.54} ) tan ( {2.5}^{'} ) \times P P I}{S} ,$$

which may yield the iOS point height and width required for a 0.0 log MAR optotype at a given viewing distance D.

In some embodiments, a Static Visual Acuity (SVA) test may be implemented, as Step 2000. A target, or optotype, such as the letter “E,” may be displayed in various orientations (for example, up, down, left, and right), and an initial size of the target may be moderate, with adaptive adjustments made based on participant responses. The participant may be instructed to verbally identify the target's orientation as it appears, and the application may utilize a microphone and speech-recognition capabilities to capture responses. For correct responses, the target size may be decreased for subsequent trials; for incorrect responses, the target size may be increased. These adjustments may continue until the participant correctly identifies the target orientation in three out of five trials. Upon achieving threshold accuracy, the application may compute an SVA score representing the smallest target size recognized, and this value may determine the target size for subsequent tests.

In some embodiments, a Visual Processing Test (VPT) may be conducted, as Step 3000. The application may display the target at a size determined by the SVA procedure. An initial display time may be set to a moderate value (for example, approximately 70 milliseconds). The participant may verbally identify the target's orientation within the set display time, and speech-recognition services may be enabled to process the response. If the participant responds correctly within the time limit, the display time may be decreased for a subsequent trial; otherwise, the display time may be increased. The VPT may be determined as the shortest display time at which the participant can consistently recognize the target orientation in three out of five trials. A buffer time (for example, approximately 35 milliseconds) may be added to the VPT to compute a Visual Display Time (VDT=VPT+35 ms), which may be used as a target display-time parameter for final testing.

In some embodiments, a Mobile Gaze Stabilization Test (mGST) may be implemented, as Step 4000. Initial head-velocity bounds may be set (for example, approximately 60 degrees per second as a lower bound and approximately 150 degrees per second as an upper bound). The participant may be instructed to move their head side to side with increasing speed while maintaining focus on the target, and the target size and display time may be defined based on the outcomes of the SVA and VPT procedures. The participant may verbally identify target orientation at each speed. Based on performance, bounds may be adjusted: for a correct identification at the lower bound speed, the lower bound may be increased by approximately 20 degrees per second; for a failure at the lower bound speed, the upper bound may be set to the current lower bound, and the lower bound may be decreased by approximately 20 degrees per second; for a correct identification at the upper bound speed, the lower bound may be set to the current upper bound, and the upper bound may be increased by approximately 20 degrees per second; for a failure at the upper bound speed, the upper bound may be decreased by approximately 20 degrees per second. This iterative adjustment may continue until the bounds converge to a small range, determining a threshold head velocity at which visual acuity is maintained. The highest successful speed may be recorded as an mGST score reflecting the participant's gaze stability performance.

In some embodiments, the system may determine a metronome frequency corresponding to a target head velocity, and the metronome may guide participants to match the targeted head velocity during testing which can be verified via the camera of a mobile device. In some implementations, head motion may be characterized as a one-half sinusoidal yaw movement (for example, left-to-right only), with one beat corresponding to one half cycle of a sinusoidal waveform. An amplitude may be defined as a total excursion of approximately 40 degrees (for example, about 20 degrees left and about 20 degrees right). An average head velocity Vave may be defined over the half-sinusoid.

In some embodiments, a relationship between average velocity and movement duration may be expressed as

$${V}_{a v g} = \frac{2 A}{{T}_{half}} ,$$

which may be rearranged to

$${T}_{half} = \frac{2 A}{{V}_{a v g}} ,$$

where A denotes peak angular displacement and T_{half} denotes the duration of the half-cycle. A conversion from time per beat to beats per minute (BPM) may be expressed as

$$BPM = \frac{60}{{T}_{half}} = \frac{60 \times {V}_{a v g}}{2 A} .$$

For an amplitude where 2A≈40°, this relationship may simplify to:

$$B P M \approx 1.5 \times {V}_{avg} ,$$

which may be used to set metronome tempo for a given target average head velocity under the assumption of a 40-degree total movement and half-sinusoidal left-to-right beats. In some embodiments, a conversion from °/s→BPM can be shown in the following example list (assuming approximately 40° amplitude):

$$\begin{matrix}60 ° / s \to 90 BPM \\ 70 ° / s \to 105 BPM \\ 80 ° / s \to 120 BPM \\ 90 ° / s \to 135 BPM \\ 100 ° / s \to 150 BPM \\ 110 ° / s \to 165 BPM \\ 120 ° / s \to 180 BPM \\ 130 ° / s \to 195 BPM \\ 140 ° / s \to 210 BPM \\ 150 ° / s \to 225 BPM\end{matrix}$$

In some embodiments, the foregoing table may be used by the stimulus control module to select a metronome tempo that assists participants in pacing head movements to the desired average velocity during mobile gaze-stabilization testing.

In some embodiments, a data review, performed as processing data as Step 5000, and completion routine may present a session summary that displays results for each test, as Step 6000, including: SVA (baseline visual acuity), VPT (minimum display time required for target recognition), and mGST (maximum head velocity with visual stability), as shown in FIGS. 3J, 3K, and 3L. Such data can be presented on screen, stored on the device's memory, stored on a cloud storage device, sent to the clinician, fed into a machine learning algorithm, or any combinations thereof.

Different features, variations, and multiple embodiments have been shown and described in various details. What has been described in this application in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations, and other embodiments will come to mind of those skilled in the art, which are intended to be and are covered by this disclosure. It is indeed intended that the scope of this disclosure should be determined by a proper legal interpretation and construction of the disclosure, including equivalents, as understood by those of skill in the art relying upon the complete disclosure present at the time of filing.

What is claimed is:

1. A system for assessing gaze stability and vestibular function using a mobile device, the system comprising:
a mobile application configured to execute gaze-stability testing protocols;
a display module for presenting visual targets on a screen of the mobile device;
a head-movement and eye-tracking module utilizing real-time video captured via a camera of the mobile device;
a speech-recognition module configured to process verbal responses; and
a data-processing module that analyzes head-movement and visual-acuity data to determine gaze-stability metrics.

2. The system of claim 1, wherein the system enables remote patient assessments from the mobile device.

3. The system of claim 1, wherein the head-movement and eye-tracking module uses augmented-reality frameworks to calculate positions and distances of at least one of a user's head or eye.

4. The system of claim 1, wherein the gaze-stability testing protocols include:
a static-visual-acuity test to establish baseline visual acuity;
a visual-processing test to determine minimum display-time thresholds; and
a mobile gaze-stabilization test to evaluate a maximum head velocity at which visual acuity is maintained.

5. The system of claim 1, wherein the display module dynamically adjusts at least one of a size and an orientation of each visual target based on user performance during testing.

6. The system of claim 1, further comprising a telehealth interface that enables clinicians to access real-time test results.

7. The system of claim 1, further comprising a telehealth interface that enables clinicians to guide treatment remotely.

8. The system of claim 1, wherein the mobile application implements a modified threshold determination procedure to determine thresholds for both visual acuity and head velocity.

9. The system of claim 1, wherein the speech-recognition module uses native mobile device frameworks to process verbal responses regarding target orientation.

10. The system of claim 1, wherein the display module is configured to present a visual target at a randomized onset time within a velocity-gated window and for a presentation duration equal to a visual display time derived from a visual processing test.

11. The system of claim 1, wherein the display module dynamically scales a visual target based on a measured device-to-eye distance and device display characteristics.

12. The system of claim 11, wherein the device-to-eye distance is estimated by averaging distances from the camera to each eye derived from face-tracking geometry.

13. The system of claim 1, wherein, the mobile application configured to perform at least one of functional vestibular function testing measurements, dynamic visual acuity testing, oculomotor testing measurements, and collection of patient-reported outcomes related to vestibular function.

14. A method for evaluating vestibular function using a mobile device, comprising:
displaying a visual target on a screen of the mobile device;
guiding a user through head-movement exercises while capturing, by a camera of the mobile device, a real-time video stream of the user;
tracking head and eye movements using the video stream, using a head-movement and eye-tracking module;
collecting verbal responses via a speech-recognition module;
analyzing head-movement and visual-acuity data to determine gaze-stability metrics; and
transmitting test results for remote evaluation.

15. The method of claim 14, wherein the visual target is a dynamically adjustable optotype that is displayed for a duration based on user performance in prior tests and whose size is preset to a value relative to the user's baseline static visual acuity.

16. The method of claim 14, wherein the head-movement and eye-tracking module uses augmented-reality frameworks to calculate positions and distances of at least one of a user's head or eye.

17. The method of claim 14, wherein the head-movement exercises include at least one gaze-stability testing protocols including:
a static-visual-acuity test to establish baseline visual acuity;
a visual-processing test to determine minimum display-time thresholds; and
a mobile gaze-stabilization test to evaluate a maximum head velocity at which visual acuity is maintained.

18. The method of claim 14, further comprising
dynamically adjusting at least one of a size and an orientation of each visual target based on user performance during testing.

19. The method of claim 14, further comprising,
implementing a modified threshold determination procedure to determine thresholds for both visual acuity and head velocity of the user.

20. The method of claim 14, further comprising,
presenting the visual target at a randomized onset time within a velocity-gated window and for a presentation duration equal to a visual display time derived from a visual processing test.

21. The method of claim 14, further comprising,
dynamically scaling a visual target based on a measured device-to-eye distance and device display characteristics.

22. The method of claim 14, the method further comprises at least one of functional vestibular function testing measurements, dynamic visual acuity testing, oculomotor testing measurements, and collection of patient-reported outcomes related to vestibular function.
